(19) United States
(12) Patent Application Publication
(10) Pub. No.: US 2011/0189279 A1
(43) Pub. Date: Aug. 4, 2011

(54) PHARMACEUTICAL COMPOSITIONS WITH MODIFIED RELEASE PROPERTIES COMPRISING 5-CHLORO-N-(-METHYL)-2-THIOPHENCARBOXAMID

(75) Inventors: Katrin Rimkus, Iserlohn (DE); Frank Muskulus, Laupheim (DE); Sandra Brueck, Ottenhofen (DE); Jana Paetz, Bonn (DE)

(73) Assignee: RATIOPHARM GMBH, Ulm (DE)

(21) Appl. No.: 13/058,323

(22) PCT Filed: Aug. 10, 2009
(86) PCT No.: PCT/EP2009/005799

§ 371 Date: Apr. 22, 2011

(30) Foreign Application Priority Data
Aug. 11, 2008 (EP) 08014306.8
Apr. 7, 2009 (EP) 09005112.9

(51) Int. Cl. A61K 9/28; A61K 31/5377; A61P 7/02

(52) U.S. Cl. 424/465; 514/230.8; 424/474; 424/472

(57) ABSTRACT

The invention relates to pharmaceutical compositions with modified release properties comprising 5-Chloro-N-({(5S)-2-oxo-3-[4-(3-oxo-4-morpholinyl)-phenyl]-1,3-oxazolidin-5-yl}-methyl)-2-thiophencarboxamid and process of preparing such compositions.

The invention relates to pharmaceutical compositions with modified release properties comprising 5-Chloro-N-({(5S)-2-oxo-3-[4-(3-oxo-4-morpholinyl)-phenyl]-1,3-oxazolidin-5-yl}-methyl)-2-thiophencarboxamid and process of preparing such compositions.

5-Chloro-N-({(5S)-2-oxo-3-[4-(3-oxo-4-morpholinyl)-phenyl]-1,3-oxazolidin-5-yl}-methyl)-2-thiophencarboxamid is a low-molecular, orally administrable inhibitor of the blood coagulation factor Xa, investigated for the prophylaxis and treatment of various thrombo-embolic diseases (see WO 01/47919) and known under the INN rivaroxaban. The 5-Chloro-N-({(5S)-2-oxo-3-[4-(3-oxo-4-morpholinyl)-phenyl]-1,3-oxazolidin-5-yl}-methyl)-2-thiophencarboxamid has the following chemical structure.

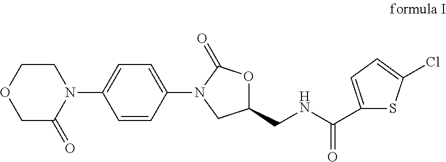

The compounds according to formula I will be hereinafter referred to as “Compound I”. In this regard it is noted that the terms “Compound I” or “compound according to formula I” refer to 5-Chloro-N-({(5S)-2-oxo-3-[4-(3-oxo-4-morpholinyl)-phenyl]-1,3-oxazolidin-5-yl}-methyl)-2-thiophencarboxamid and its solvates and hydrates as well as pharmaceutical acceptable salts thereof, preferably obtained according to the procedures as outlined in WO 01/47919. This form has been described in WO 2007/039132 as crystalline form I.

Compound I has only limited solubility in water, causing problems regarding dissolution of the API from the pharmaceutical composition, the oral bioavailability and the reproducibility of the dissolution profile in modified release formulations.

In order to improve the bioavailability of Compound I, several concepts have been put forward. WO 2005/060940 teaches the use of the wet granulation technique in combination with the use of solubilizers in order to hydrophilize the Compound I and to improve bioavailability.

WO 2007/039122 discloses immediate release forms comprising the use of an amorphous or semi-stable crystalline modification of Compound I as API. The use of these modifications significantly increases the solubility and the oral bioavailability compared to the formulations described in WO2005/060940, using the Compound I in crystalline modification I.

WO 2006/072367 describes formulations with modified release properties. The formulations therein comprises compound I in the hydrophilized crystalline modification I according to in WO 2005/060940 or in the amorphous form according to WO 2007/039132 in combination with erosion-matrix systems and osmotic release systems. In the case of an osmotic release system, tablets are enveloped by a semi-permeable membrane which has at least one orifice. The semi-permeable membrane is impermeable to the components of the core but permits water to enter the system from outside by osmosis. The water which penetrates in, then releases through the osmotic pressure produced the active ingredient in dissolved or suspended form from the orifice(s) in the membrane.

Furthermore, the use of erosion-matrix systems is generally hampered by several facts strongly related to its mechanism of action. The released erosion matrix is resorbed by the organism and therefore the erosion-matrix itself could result in side effects. The properties of the polymer are often pH-dependent which could result in strong variety of the release depending on the fasting state of the patient for example or with the nutrition taken in connection with taking of the drug. Furthermore interactions with the gastro-intestinal motility occur. The final 25% of the dosage is often released in an uncontrolled manner since the tablets finally dissolve by crumbling. Finally there is a high dependency of the release properties form the polymer cross-linking which could only be described within wide ranges by the suppliers.

Using the stable crystalline form I Rivaroxaban in erosion-matrix systems or in osmotic systems the reduced dissolution rate observed in the examples of WO2006/072367 are mainly caused by the slow dissolution of the agent itself and not by the osmotic system. In addition release from such dosage forms is incomplete and might finally result in a significant amount of drug which is not administered to the patient in the expected period of time. Such an interaction could result in a very unpredictable in-vivo release of the agent and subsequently could cause adverse events and toxicity effects or insufficient efficacy.

Employing the above hydrophilization by wet granulation approach, using the stable crystalline modification Compound I, does not provide sufficient bioavailability compared to using the amorphous state according to the teaching in WO2007/039122. The use of Compound I in the amorphous state is hampered by stability issues due to the tendency of the amorphous form to switch to a semi-crystalline state. The wet granulation technique furthermore is energy and time-consuming and cost-intensive.

It is therefore an object of the invention to provide a pharmaceutical composition with modified release properties comprising Compound I or a pharmaceutically acceptable salt thereof which does not encounter the above mentioned problems. Preferably, a pharmaceutical composition should be provided having improved properties like solubility, dissolution profile, well-defined, predictable and reproducible dissolution rates, stability, flowability and bioavailability. In particular, a modified release dosage form should be provided, wherein the drug is completely released after 24 hours. Such an oral dosage form should be producible in a large scale in an economic beneficial way.

It has now been found that the above problems can be overcome by providing pharmaceutical formulations and pharmaceutical dosage forms with modified release properties comprising Compound I as active ingredient and a solubilizer, optionally a pseudo-emulsifier, a non-erodible polymer and optionally a pore-forming substance as excipients.

The problem can be further overcome by specific processes for the manufacture of a pharmaceutical formulation and pharmaceutical dosage forms of Compound I or its solvates and hydrates.

The release modifying properties of the formulations of the present invention are introduced by using suitable “modified release systems” comprising non-erodible polymers and preferably pore-forming substances.

Preferably, the used “modified release system” is capable of increasing the dissolution time of the pharmaceutical composition at least fourfold, more preferably at least eightfold, according to USP release method using apparatus 2 (paddle), compared to the same pharmaceutical composition without the release modifying system.

Hence, a subject of the present invention is a pharmaceutical composition with modified release properties comprising

(a) a compound according to formula I as active ingredient

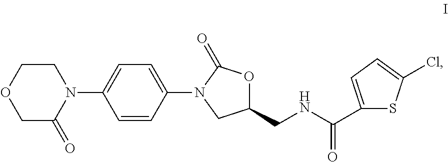

its solvates, hydrates and/or pharmaceutically acceptable salts,
(b) a solubilizer,
(c) optionally a pseudo-emulsifier,
(d) a non-erodible polymer, preferably a non-erodible polymer having a water solubility of 10 mg/l or less at a temperature of 25° C., and
(e) preferably a pore-forming substance, having a water solubility of more than 100 mg/l at a temperature of 25° C.

Preferably, components (d) and (e) constitute a “modified release” system, which determines the drug release properties of the formulation. Alternatively, also component (d) alone can constitute the modified release system. Furthermore, it is also preferred that the modified release system further comprises a plasticizer (f) as illustrated in detail below. Hence, the modified release system comprises or consists of the components

(d) or
(d) and (e) or
(d) and (f) or
(d) and (e) and (f).

In the pharmaceutical composition of the present invention Compound I as the active ingredient (=component (a)) preferably is present in crystalline form, wherein the crystalline modification I as described in WO 01/47919 is particularly preferred. Preferably, the active ingredient is present in the form of the free base.

In a preferred embodiment the active ingredient (a) is employed in a micronized form. That means, the active ingredient (a) of the pharmaceutical composition of the present invention (=Compound I) has a volume mean particle size (D₅₀) of 0.1 to 100 μm, more preferably of 0.3 to 50 μm, further more preferably of 1 to 20 μm, most preferably of 2 to 10 μm. The volume mean particle size (D₅₀) is determined by the light scattering method, using a Mastersizer 2000 apparatus made by Malvern Instruments (wet measurement, 2000 rpm, ultrasonic waves for 60 sec., data interpretation via Fraunhofer method).

The pharmaceutical composition further comprises one or more solubilizers (b). Generally, the term “solubilizer” means any organic excipient, which improves the solubility and dissolution of the active pharmaceutical ingredient. Preferably, the solubilizer is capable of reducing the dissolution time of a pharmaceutical composition by 5%, more preferably by 20%, according to USP release method using apparatus 2 (paddle), compared to the same pharmaceutical composition comprising calcium hydrogen phosphate instead of the solubilizer.

The solubilizers are selected, for example, from the group of known inorganic or organic excipients. Such excipients preferably include polymers, low molecular weight oligomers, natural products and surfactants.

Preferably the solubilizer is a water-soluble compound having a water solubility of more than 10 mg/l, more preferably of more than 20 mg/l, still more preferably of more than 50 mg/l at a temperature of 25° C. The solubility of the solubilizer might be e.g. up to 1000 mg/l at a temperature of 25° C. The water-solubility is determined according to the column elution method of the Dangerous Substances Directive (67/548/EEC), Annex V, Chapter A6.

In a preferred embodiment the solubilizer is a hydrophilic polymer preferably having the above mentioned water-solubility. Generally, the term “hydrophilic polymer” encompasses polymers comprising polar groups. Examples for polar groups are hydroxy, amino, carboxy, carbonyl, ether, ester, sulfonate. Hydroxy groups are particularly preferred.

The hydrophilic polymer usually has a weight average molecular weight ranging from 1,000 to 250,000 g/mol, preferably from 2,000 to 100,000 g/mol, particularly from 4000 to 50,000 g/mol. Furthermore, a 2% w/w solution of the hydrophilic polymer in pure water preferably has a viscosity of from 2 to 8 mPas at 25° C. The viscosity is determined according to the European Pharmacopoeia (hereinafter referred to as Ph. Eur.), 6^{th} edition, chapter 2.2.10.

Furthermore, the hydrophilic polymer used as solubilizer preferably has a glass transition temperature (Tg) or a melting point of 25° C. to 150° C., more preferably of 40° C. to 100° C. The glass transition temperature, Tg, is the temperature at which the hydrophilic polymer becomes brittle on cooling and soft on heating. That means, above Tg the hydrophilic polymers become soft and capable of plastic deformation without fracture. The glass transition temperature or the melting point are determined with a Mettler-Toledo® DSC 1, wherein a heating rate of 10° C. per minute and a cooling rate of 15° C. per minute is applied.

Examples for suitable hydrophilic polymers useful as solubilizer are derivatives of cellulose, hydrophilic derivatives of cellulose (hydroxyproplymethyl cellulose (HPMC), hydroxypropyl cellulose (HPC), carboxymethyl cellulose (CMC), preferably sodium or calcium salts thereof, hydroxyethyl cellulose, hydroxypropyl cellulose (HPC), polyvinylpyrrolidone, preferably having an average molecular weight of 10,000 to 60,000 g/mol, copolymers of polyvinylpyrrolidones, preferably copolymers comprising vinylpyrrolidone and vinylacetate units (e.g. Povidon® VA 64; BASF), preferably having a weight average molecular weight of 40,000 to 70,000 g/mol, polyoxyethylene-alkylethers, polyethylene glycol, co-blockpolymers of ethylene oxide and propylene oxide (Poloxamer, Pluronic®), derivates of methacrylates, polyvinylalcohol and/or polyethylene glycols or derivatives thereof. The weight average molecular weight is preferably determined by gel electrophoresis.

Furthermore, derivates of glycerol, derivates of dextrins, and derivates of fatty acids, e.g. sodium lauryl sulfate, can be used as solubilizers.

Moreover, sugar alcohols like isomalt, sorbitol, xylitol or mannitol can be used as solubilizers.

In particular, cellulose derivatives (especially hydroxypropylmethyl cellulose (HPMC) and/or hydroxypropyl cellulose (HPC)), sugar alcohols (especially isomalt), polyvinylpyrrolidone and copolymers of polyvinylpyrrolidone are used as solubilizer.

It is particularly preferred that the above mentioned kinds of hydrophilic polymers fulfill the functional requirements (molecular weight, viscosity, melting point, non-semi-permeable properties) as illustrated above. Preferably, the term “solubilizer” does not comprise microcrystalline cellulose.

In the pharmaceutical composition of the present invention at least one of the above-mentioned solubilizers is present. Alternatively, a combination of two or more solubilizers can be employed.

The pharmaceutical composition optionally further comprises one or more pseudo-emulsifiers (c). Generally, the term “pseudo-emulsifier” means any organic excipient, which avoids an agglomeration of a micronized active ingredient (API) after disintegration of the pharmaceutical composition, in order to improve the solubility of the active ingredient.

The pseudo-emulsifiers preferably are selected from natural products, more preferably from natural gums. Natural gums are polysaccharides of natural origin, capable of causing a viscosity increase in solution, even at concentrations less than 15%. Generally, the addition of 5 wt.-% of the pseudo-emulsifiers—preferably of the natural gum—to an aqueous solution causes a viscosity increase of said solution of at least 1%, preferably of at least 2%, especially of at least 5%. Examples for suitable natural gums are

Agar (E406), preferably obtained from seaweed,
Alginic acid (E400), preferably obtained from seaweed,
Beta-glucan, preferably from obtained oat or barley bran,
Carrageenan (E407), preferably obtained from seaweed,
Chicle gum, preferably obtained from the chicle tree,
Dammar gum, preferably obtained from the sap of Dipterocarpaceae trees,
Gellan gum (E418), preferably produced by bacterial fermentation,
Glucomannan (E425), preferably obtained from the konjac plant,
Gum arabica (E414), preferably obtained from the sap of acacia trees,
Gum ghatti, preferably obtained from the sap of Anogeissus trees,
Gum tragacanth (E413), preferably obtained from the sap of Astragalus shrubs,
Karaya gum (E416), preferably obtained from the sap of sterculia trees,
Locust bean gum (E410), preferably obtained from the seeds of the carob tree,
Mastic gum, preferably obtained from the mastic tree,
Psyllium seed husks, preferably obtained from the Plantago plant,
Sodium alginate (E401), preferably obtained from seaweed,
Spruce gum, preferably obtained from spruce trees,
Tara gum (E417), preferably obtained from the seeds of the tara tree.

Furthermore, the pseudo-emulsifier can be selected from phospholipids, preferably lecithin. Moreover, the pseudo-emulsifier can comprise proteins, preferably phosphoproteins like casein.

In a preferred embodiment the pseudo-emulsifier comprises gum arabica, agar and/or lecithin, in particular gum arabica.

In the pharmaceutical composition of the present invention at least one of the above-mentioned pseudo-emulsifiers may be present. Alternatively, a combination of two or more pseudo-emulsifiers can be employed. During the dissolution of the formulation, the combination of a solubilizer and a pseudo-emulsifier usually is aimed to reduce the agglomeration of the particles during the dissolution and increase the effect of the solubilizers. The mechanism of action of the pseudo-emulsifier usually mainly relies on an enhancement of viscosity. However pseudo-emulsifiers also possess emulsifying properties.

The pharmaceutical composition of the present invention further comprises a non-erodible polymer (d). Preferably, the non-erodible polymer has a water solubility of 10 mg/l or less at a temperature of 25° C., more preferably of 8 mg/l or less, especially from 0.01 to 5 mg/l. The water-solubility is determined according to the column elution method of the Dangerous Substances Directive (67/548/EEC), Annex V, Chapter A6.

The non-erodible polymer usually has a weight average molecular weight ranging from more than 50,000 to 2,500,000 g/mol, preferably from more than 250,000 to 2,000,000 g/mol, particularly from 400,000 to 1,500,000 g/mol. Furthermore, a 2% w/w solution of the non-erodible polymer in pure water preferably has a viscosity of more than 2 mPas, more preferably of more than 5 mPas, particularly more than 8 mPas and up to 850 mPas when measured at 25° C. The viscosity is determined according to Ph. Eur., 6^{th} edition, chapter 2.2.10. In the above definition the term “solution” may also refer to a partial solution (in case that the polymer does not dissolve completely in the solution). The weight average molecular weight is preferably determined by gel electrophoresis.

It is further preferred that the non-erodible polymer has a melting temperature of below 220° C., more preferably of between 25° C. and 200° C. In a particularly preferred embodiment the melting temperature is between 35° C. and 190° C. The determination of the melting temperature is carried out according to Ph. Eur., 6^{th} edition, chapter 2.2.15.

Preferably, the non-erodible polymer is selected from methacrylates, e.g. Eudragit® NE, Eudragit® RS/RL (Evonik); cellulose derivatives, e.g. ethyl cellulose and cellulose acetate phthalate; polyvinyl alcohol or derivatives thereof; polyvinyl acetate or derivatives thereof; polyvinyl chloride or derivatives thereof; shellac and mixtures thereof. Eudragit® NE is an ethylacrylate/methylacrylate co-polymer and Eudragit® RS/RL is an acrylate/methacrylate co-polymer with a low content of quaternary ammonium groups.

To summarize, the following kinds of non-erodible polymers are particularly preferred.

1. Cellulose ether, preferably ethyl cellulose, preferably ethyl cellulose having an average molecular weight of 150,000 to 300,000 and/or an average degree of substitution, ranging from 2,2 to 2.6;
2. cellulose ester, preferably cellulose acetate phthalate, carboxymethylethyl cellulose, hydroxypropylmethyl cellulose phthalate;
3. copolymers of methacrylic acid or methacrylic acid esters, preferably ethylacrylate-methylmethacrylate-trimethylammonioethylmethacrylate-chloride 1:2:0,1 (Eudragit® RS), ethylacrylate-methylmethacrylate-trimethylammonioethylmethacrylate-chloride 1:2:0,2 (Eudragit® RL), ethylacrylate-methylmethacrylate 2:1 (Eudragit® NE), methacrylic acid-methylmethacrylate, wherein the weight ratio is 1:2 (Eudragit® S), methacrylic acid-methylmethacrylate, wherein the weight ratio is 1:1 (Eudragit® L);
4. polyvinylacetate or polyvinyl acetate copolymers, preferably polyvinyl acetate phthalate; and mixtures thereof.

It is particularly preferred that the above mentioned kinds of non-erodible polymers fulfill the functional requirements (molecular weight, viscosity, melting point, non-semi-permeable properties) as illustrated above. Hence, cellulose acetate is not regarded as a non-erodible polymer, since the use of cellulose acetate usually leads to a shell having semi-permeable properties. Furthermore, cellulose acetate has a melting point of about 260° C. Analogously, microcrystalline cellulose (melting point of about 230° C.) is not regarded as a non-erodible polymer.

The pharmaceutical composition of the present invention further preferably comprises one or more pore-forming substances (e). The pore-forming substances usually are water soluble, allow the entrance of water and enable a swelling of the non-erodible polymer, and thus enable the release of compound I (=component (a)) from the polymer.

The pore-forming substance preferably has a water solubility of more than 100 mg/l at a temperature of 25° C., more preferred of more than 250 mg/l and particularly preferred of more than 25 g/l. The water-solubility of the pore-forming substance may range up to 2.5 kg/l. The water-solubility is determined according to the column elution method of the Dangerous Substances Directive (67/548/EEC), Annex V, Chapter A6.

The pore-forming substances can be selected from inorganic substances, preferably from inorganic salts such as NaCl, KCl, Na₂SO₄. Furthermore, the pore-forming substance can be selected from organic substances, in particular from organic substances being solid at 30° C. and having the above-mentioned water solubility. Suitable examples are PEG, particularly PEG having a weight average molecular weight of from 2,000 to 10,000 g/mol.

Furthermore, povidone (polyvinylpyrrolidone), preferably having a weight average molecular weight of from 5,000 to 30,000 g/mol, PEG with a weight average molecular weight of 380-4800, polyethylene oxide with a weight average molecular weight of less than 100,000 and a viscosity of less than 20 mPa*s, sugar alcohols like mannitol, sorbitol, xylitol, isomalt, anorganic salts like sodium chloride are also suitable as pore-forming substances.

Furthermore, in a preferred embodiment the pharmaceutical composition of the present invention further comprises one or more plasticizers (f). The “plasticizers” usually are compounds capable of lowering the glass transition temperature (T_{g}) of the non-erodible polymer, preferably of lowering T_{g} from 1 to 50° C., especially from 5 to 30° C. Plasticizers (f) usually are low molecular weight compounds (having a molecular weight from 50 to 500 g/mol) and comprise at least one hydrophilic group.

Examples of suitable plasticizers are dibutyl sebacetate (DBS), Myvacet® (acetylated monoglycerides), triacetin (GTA), citric acid esters, like acetyltriethyl citrate (ATEC) or triethyl citrate (TEC), propylene glycol, dibutyl phathalate, diethyl phathalate, or mixtures thereof.

The combined use of the non-erodible polymer (d) and the pore-forming substance (e) and optionally the plasticizer (f) preferably is capable of modifying the drug release rate.

Preferred combinations of solubilizer, pseudo-emulsifier (only optional), non-erodible polymer and pore forming substance are:

Polyvinylpyrrolidone/gum arabica/acrylate based polymer/PEG,
copolymers of polyvinylpyrrolidone/gum arabica/acrylate based polymer/PEG,
hydroxypropylmethyl cellulose (HPMC)/gum arabica/acrylate based polymer/PEG,
copolymers of polyvinylpyrrolidone and HPMC/gum arabica/acrylate based polymer/PEG,
hydroxypropyl cellulose (HPC)/gum arabica/acrylate based polymer/PEG,
polyvinylpyrrolidone/agar/acrylate based polymer/PEG,
copolymers of polyvinylpyrrolidone/agar/acrylate based polymer/PEG,
copolymers of polyvinylpyrrolidone, sodium lauryl sulfate/agar/acrylate based polymer/PEG,
hydroxypropylmethyl cellulose (HPMC)/agar/acrylate based polymer/PEG,
copolymers of polyvinylpyrrolidone and HPMC/agar/acrylate based polymer/PEG,
hydroxypropyl cellulose (HPC)/agar/acrylate based polymer/PEG,
polyvinylpyrrolidone/lecithin/acrylate based polymer/PEG,
copolymers of polyvinylpyrrolidone/lecithin/acrylate based polymer/PEG,
hydroxypropylmethyl cellulose (HPMC)/lecithin/acrylate based polymer/PEG,
copolymers of polyvinylpyrrolidone and HPMC/lecithin/acrylate based polymer/PEG,
hydroxypropyl cellulose (HPC)/lecithin/acrylate based polymer/PEG,
isomalt/gum arabica/acrylate based polymer/PEG,
isomalt/agar/acrylate based polymer/PEG,
isomalt/lecithin/acrylate based polymer/PEG,
isomalt/carrageenan/acrylate based polymer/PEG,
polyvinylpyrrolidone/gum arabica/ethylcellulose/PEG,
copolymers of polyvinylpyrrolidone/gum arabica/ethylcellulose/PEG,
hydroxypropylmethyl cellulose (HPMC)/gum arabica/ethylcellulose/PEG,
copolymers of polyvinylpyrrolidone and HPMC/gum arabica/ethylcellulose/PEG,
hydroxypropyl cellulose (HPC)/gum arabica/ethylcellulose/PEG,
polyvinylpyrrolidone/agar/ethylcellulose/PEG,
copolymers of polyvinylpyrrolidone/agar/ethylcellulose/PEG,
copolymers of polyvinylpyrrolidone, sodium lauryl sulfate/agar/ethylcellulose/PEG,
hydroxypropylmethyl cellulose (HPMC)/agar/ethylcellulose/PEG,
copolymers of polyvinylpyrrolidone and HPMC/agar/ethylcellulose/PEG,
hydroxypropyl cellulose (HPC)/agar/ethylcellulose/PEG,
polyvinylpyrrolidone/lecithin/ethylcellulose/PEG,
copolymers of polyvinylpyrrolidone/lecithin/ethylcellulose/PEG,

hydroxypropylmethyl cellulose (HPMC)/lecithin/ethylcellulose/PEG,

copolymers of polyvinylpyrrolidone and HPMC/lecithin/ethylcellulose/PEG,
hydroxypropyl cellulose (HPC)/lecithin/ethylcellulose/PEG,
isomalt/gum arabica/ethylcellulose/PEG,
isomalt/agar/ethylcellulose/PEG,
isomalt/lecithin/ethylcellulose PEG and/or
isomalt/carrageenan/ethylcellulose/PEG.

Polyvinylpyrrolidone/gum arabica/acrylate based polymer/povidone,

copolymers of polyvinylpyrrolidone/gum arabica/acrylate based polymer/povidone,
hydroxypropylmethyl cellulose (HPMC)/gum arabica/acrylate based polymer/povidone,
copolymers of polyvinylpyrrolidone and HPMC/gum arabica/acrylate based polymer/povidone,
hydroxypropyl cellulose (HPC)/gum arabica//acrylate based polymer/povidone,
polyvinylpyrrolidone/agar, /acrylate based polymer/povidone,
copolymers of polyvinylpyrrolidone/agar/acrylate based polymer/povidone,
copolymers of polyvinylpyrrolidone, sodium lauryl sulfate/agar/acrylate based polymer/povidone,
hydroxypropylmethyl cellulose (HPMC)/agar/acrylate based polymer/povidone,
copolymers of polyvinylpyrrolidone and HPMC/agar/acrylate based polymer/povidone,
hydroxypropyl cellulose (HPC)/agar/acrylate based polymer/povidone,
polyvinylpyrrolidone/lecithin/acrylate based polymer/povidone,
copolymers of polyvinylpyrrolidone/lecithin/acrylate based polymer/povidone,
hydroxypropylmethyl cellulose (HPMC)/lecithin/acrylate based polymer/povidone,
copolymers of polyvinylpyrrolidone and HPMC/lecithin/acrylate based polymer/povidone,
hydroxypropyl cellulose (HPC)/lecithin/acrylate based polymer/povidone,
isomalt/gum arabica/acrylate based polymer/povidone,
isomalt/agar/acrylate based polymer/povidone,
isomalt/lecithin/acrylate based polymer/povidone,
isomalt/carrageenan//acrylate based polymer/povidone,
Polyvinylpyrrolidone/gum arabica/acrylate based polymer/NaCl,
copolymers of polyvinylpyrrolidone/gum arabica/acrylate based polymer/NaCl,
hydroxypropylmethyl cellulose (HPMC)/gum arabica/acrylate based polymer/NaCl,
copolymers of polyvinylpyrrolidone and HPMC/gum arabica/acrylate based polymer/NaCl,
hydroxypropyl cellulose (HPC)/gum arabica/acrylate based polymer/NaCl,
polyvinylpyrrolidone/agar/acrylate based polymer/NaCl,
copolymers of polyvinylpyrrolidone/agar/acrylate based polymer/NaCl,
copolymers of polyvinylpyrrolidone, sodium lauryl sulfate/agar/acrylate based polymer/NaCl,
hydroxypropylmethyl cellulose (HPMC)/agar/acrylate based polymer/NaCl,
copolymers of polyvinylpyrrolidone and HPMC/agar/acrylate based polymer/NaCl,
hydroxypropyl cellulose (HPC)/agar/acrylate based polymer/NaCl,
polyvinylpyrrolidone/lecithin/acrylate based polymer/NaCl,
copolymers of polyvinylpyrrolidone/lecithin/acrylate based polymer/NaCl,
hydroxypropylmethyl cellulose (HPMC)/lecithin/acrylate based polymer/NaCl,
copolymers of polyvinylpyrrolidone and HPMC/lecithin/acrylate based polymer/NaCl,
hydroxypropyl cellulose (HPC)/lecithin/acrylate based polymer/NaCl,
isomalt/gum arabica/acrylate based polymer/NaCl,
isomalt/agar/acrylate based polymer/NaCl,
isomalt/lecithin/acrylate based polymer/NaCl,
isomalt/carrageenan//acrylate based polymer/NaCl,

Alternatively, also the above mentioned combinations comprising two out of four or three out of four components are suitable.

Preferred combinations of components (d) and (f) are as follows:

Ethyl cellulose/dibutyl sebacetate (DBS), ethyl cellulose/Myvacet® (acetylated monoglycerides), ethyl cellulose/triacetin (GTA), ethyl cellulose/acetyltriethyl citrate (ATEC) ethyl cellulose/triethyl citrate (TEC), polyvinylacetate/triethyl citrate (TEC) or polyvinylacetate propylene glycol. In case of polymethacrylates as component (d), preferably no plasticizer (f) is added.

Generally, in the pharmaceutical composition of the present invention the active ingredient (a) can be present in an amount of 1 to 90 wt.-%, preferably 4 to 60 wt.-%, more preferably 5 to 40 wt.-%, and particularly preferred between 6 and 20 wt.-%, based on the total weight of the composition.

Generally, in the pharmaceutical composition of the present invention the solubilizer (b) can be present in an amount of 0.1 to 75 wt.-%, preferably 1 to 60 wt.-%, more preferably 5 to 30 wt-%, based on the total weight of the composition.

In a preferred embodiment the weight ratio of active ingredient (a) to solubilizer (b) is 1:15 to 20:1, more preferably 1:10 to 10:1, in particular 1:3 to 3:1.

Generally, in the pharmaceutical composition of the present invention the pseudo-emulsifier (c) can be present in an amount of 0 to 15 wt.-%, preferably 0.1 to 10 wt. %, more preferably 0.5 to 5 wt.-%, based on the total weight of the composition. It has been found that a higher amount of pseudo-emulsifier in the composition might result in an incomplete drug release. Therefore, it is preferred that the pharmaceutical composition of the present invention does not comprise more than 15 wt.-% of pseudo-emulsifier, more preferably not more than 10 wt.-%, particularly not more than 5%. It is preferred that the pharmaceutical composition of the present invention does not comprise more than 15 wt.-% of pseudo-emulsifier, more preferably not more than 10 wt.-%, particularly not more than 5%. Especially it is preferred that the pharmaceutical composition of the present invention does not comprise more than 15 wt.-% of a natural gum, more preferably not more than 10 wt.-%, particularly not more than 5%.

The “release modifying system” comprising components (d) and optionally (e) may be present in an amount of 5-50 wt.-%, more preferably in an amount of 10-40 wt.-%, based on the total weight of the pharmaceutical composition of the present invention. Alternatively, the “release modifying system”, comprising components (d), (e) and (f), may be present in an amount of 5-50 wt.-%, more preferably in an amount of 10-40 wt.-%, based on the total weight of the pharmaceutical composition of the present invention. Plasticizer (f) may be present in an amount of 0 to 25 wt. %, preferably from 1 to 15 wt.-%, based on the total weight of the pharmaceutical composition.

The weight ratio of components (d) to (e) may range from 1:1 to 50 to 1. However, in order to achieve the desired above mentioned release properties, the weight ratio of components (d) to (e) preferably is from 2:1 to 10:1 or 3:1 to 20:1, more preferably 5:1 to 15:1.

If a plasticizer (f) is used, component (f) usually is present in an amount of 1 to 30 wt. % (especially in the case of ethyl cellulose as component (d)), preferably 2 to 15 wt. % (especially in case of polyvinyl acetate as component (d)), based on the combined weight of components (d) and (f).

In a preferred embodiment the pharmaceutical composition of the present invention is in the form of a tablet comprising a core and a shell, wherein the core comprises components (a), (b) and optionally (c) and wherein the release modifying shell comprises components (d) and optionally (e) and optionally (f).

Generally, due to the nature of pharmaceutical excipients, it cannot be excluded that a certain compound meets the requirements of more than one of the components (b) to (e) of the pharmaceutical composition of the present invention. However, in order to enable an unambiguous distinction it is preferred in the present application that one and the same pharmaceutical excipient can only function as one of the compounds (b) or (c) in the core and as one of the components (d) and (e) in the shell. For example, if mannitol functions as solubilizer (b) in the core, it cannot additionally function as pseudo-emulsifier. However, in this case, mannitol may function as pore-forming substance (e) in the shell, wherein said function as pore-forming substance automatically excludes its function as component (d) (irrespective that mannitol is not a non-erodible polymer). Furthermore, in the present application rivaroxaban only functions as component (a) but not as one of components (b) to (e).

Hence, a further subject of the present invention is a tablet, comprises a core and a shell, wherein the core comprises components (a), (b) and optionally (c) and wherein the shell comprises components (d) and (e).

In this embodiment the non-erodible polymer (d) consists of compounds which do not form a semi-permeable membrane. That means, the non-erodible polymer does not form a coating which is essentially impermeable to the components (a), (b) and optionally (c) of the core but permits water to enter the system from outside by osmosis. Contrary, the non-erodible polymer forms a coating which is permeable for the components (a), (b) and optionally (c). The release follows the mode of action per diffusion according the “Ficksche Gesetze”

By application of an additional pore former (e) the components (a), (b) and optionally (c) can diffuse also through the pores generated by dissolving the pore former.

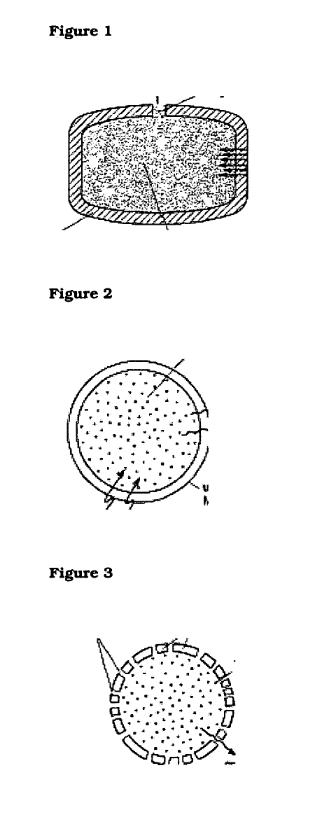

The different modes of action of the systems according to the prior art and according to the present invention is illustrated in FIGS. 1 to 3.

FIG. 1 illustrates an osmotic system as described in WO 2006/072367.

FIG. 2 illustrates retardation by a coating system using a non erodible polymer.

FIG. 3 illustrates retardation by a coating system according to the present invention using a non-erodible polymer together with a pore former.

Detailed explanations about the different modes of action can be found in “Pharmazeutische Technologie” Sucker, Fuchs, Speiser.

The tablet of the present invention can be prepared by specific processes.

Generally, a process for producing a tablet according to the present invention containing core and release modifying shell, comprises the steps of
(i) mixing components (a), (b) and optionally (c) and/or further excipients,
(iv) compressing the mixture into tablets, and
(v) coating the tablets with a coating, comprising compounds (d) and optionally (e) and optionally (f).

In step (i) the compound according to formula I (=Compound I) is mixed with excipients. The mixing process can be carried out in conventional mixers, e.g. in a free fall mixer like Turbula® T 10B (Bachofen AG, Switzerland).

Preferably, the excipients comprise a solubilizer and a pseudo-emulsifier. Generally, it is noted that all comments made above regarding the solubilizer (b) and the pseudo-emulsifier (c) of the pharmaceutical composition of the present invention also apply for the processes of the present invention.

In the process of the present invention (instead or preferably in addition to solubilizer and pseudo-emulsifier) one or more further pharmaceutically acceptable excipient(s), such as fillers, lubricants, glidants, anti-sticking agents, and disintegrating agents, can be employed. Regarding the above-mentioned pharmaceutically acceptable excipients, the application refers to “Lexikon der Hilfsstoffe für Pharmazie, Kosmetik and angrenzende Gebiete”, edited by H. P. Fiedler, 4th Edition, Edito Cantor, Aulendorf and earlier editions, and “Handbook of Pharmaceutical Excipients”, Third Edition, edited by Arthur H. Kibbe, American Pharmaceutical Association, Washington, USA, and Pharmaceutical Press, London.

The pharmaceutical compositions of the present invention may comprise one or more fillers. Generally, a filler usually is a substance suitable for increasing the bulk volume of the mixture and hence increasing the size of the resulting dosage form, preferably of the resulting tablet. Preferred examples of the fillers are soluble and insoluble excipients like lactose or calcium hydrogen phosphate. The filler is for example present in an amount of 0 to 80 wt. %, preferably of 10 to 60 wt. % of the total weight of the composition.

The function of the lubricant is to ensure that tablet formation and ejection can occur with low friction between the solids and the die wall. The lubricant is preferably a stearate or fatty acid, more preferably an earth alkali metal stearate, such as magnesium stearate. The lubricant is suitably present in an amount of 0 to 2 wt. %, preferably about 0.5 to 1.5 wt. % of the total weight of the composition.

Usually, disintegrants are understood as substances capable of breaking up the tablet into small fragments when in contact with a liquid, preferably when in contact with water. Preferred disintegrating agents are croscarmellose sodium, sodium carboxymethyl starch, cross-linked polyvinylpyrrolidone (crospovidone) or sodium carboxymethyl glycolate (e.g. Explotab®), sodium bicarbonate. The disintegrating agent is suitably present in an amount of 0 to 20 wt. %, more preferably at about 1 to 15 wt. % of the total weight of the composition.

The glidant can for example be colloidal silicon dioxide (e.g. Aerosil®). Preferably the glidant agent is present in an amount of 0 to 8 wt. %, more preferably at 0.1 to 3 wt. % of the total weight of the composition.

The anti-sticking agent is for example talcum and may be present in amounts of 0 to 5%.wt, more preferably in an amount of 0.5 to 3 wt. % of the total weight of the composition.

Generally, if in the processes of the present invention solubilizers (b) or pseudo-emulsifiers (c) are used, all other excipients (e.g. fillers, binding agents, lubricants, disintegrating agents, glidants and anti-sticking agents) are defined as not comprising those compounds which were specified above as being solubilizers or pseudo-emulsifiers.

The present invention further provides two different concepts for “mixing” the active ingredient (a) and the solubilizer (b).

In a first preferred embodiment components (a) and (b) are employed in the form of a intermediate, which is obtained by blending of compounds (a) and (b).

The blending can be carried out in conventional blenders. Suitable examples are tumble blenders such as Turbula TC 10 B.

Alternatively, the intermediate comprising can be obtained by combined milling (e.g. combined micronizing) components (a) and (b).

The milling process for producing the intermediate e.g. can be carried out in a ball mill, pin mill or jet mill.

The blending and milling time may vary from 2 to 30 minutes, preferably from 5 to 20 minutes.

Preferably, the blending and/or milling conditions are chosen such that in the resulting intermediate at least 10% of the surface of the particles of component (a) are covered with solubilizer (b), more preferably at least 30%, in particular at least 50%.

In a second preferred embodiment components (a) and (b) are employed in the form of a co-precipitate, obtained by a process comprising the steps
(α) dissolving components (a) and (b) in a solvent,
(β) precipitating a complex comprising components (a) and (b) by adding an anti-solvent.

In step (α) the compound according to formula I (=Compound (a)) is dissolved together with the solubilizer (b) in a solvent. The solvent could be a pharmaceutically acceptable organic solvent or mixtures thereof. Preferably, the solvent is an alcohol or an organic acid. Most preferably, the solvent is acetic acid or ethanol.

In the second step (β) a complex, comprising a compound according to formula I and solubilizer is precipitated by adding an anti-solvent. The anti-solvent could be water or a pharmaceutically acceptable organic solvent or a mixture thereof. Preferably, the anti-solvent is water. If necessary, also a pH-shift could be employed in order to induce precipitation.

In a preferred embodiment of the intermediate or of the co-precipitate the weight ratio of active ingredient (a) to solubilizer (b) is 1:15 to 20:1, more preferably 1:10 to 10:1.

Hence, the above outlined intermediate as well as the above-outlined coprecipitate can be used in the process for producing a tablet according to the present invention containing core and release modifying shell comprising steps (i), (iv) and (v). That means, the tablets of the present invention can be prepared by a direct-compression method.

Alternatively, the tablets of the present invention can be prepared by a dry granulation method.

That means, in a preferred embodiment the above mentioned process further comprises the steps of

(ii) dry-compaction of the mixture resulting from step (i) to give a comprimate, and
(iii) granulating the comprimate and optionally adding further excipients.

In the second step (ii) the mixed formulation resulting from step (i) is subjected to a dry-compaction step in order to receive a comprimate. The dry-compaction generally is carried out in the absence of essential amounts of solvents.

In a preferred embodiment the dry-compaction step is carried out by roller compaction. Alternatively, e.g. slugging can be used. If roller compaction is applied, the compaction force usually ranges from 2 to 50 kN/cm, preferably from 5 to 45 kN/cm, more preferably from 8 to 28 kN/cm.

The gap width of the roller compactor usually is 0.8 to 5 mm, preferably 1 to 4 mm, more preferably 1.5 to 3.2 mm, especially 1.8 to 3.0 mm.

During dry-compaction the conditions are chosen such that the resulting comprimate comprises a true density of from 0.55 to 0.85, preferably from 0.6 to 0.8.

Preferably, the roller compactor is equipped with a cooling device. Usually, the comprimated pharmaceutical composition should not be subjected to temperatures above 50° C.

In a third step of the process of the present invention (iii) the comprimate (received in step (ii)) is granulated.

Preferably, the granulation step is carried out by an elevated sieving equipment, e.g. Comil® U5 (Quadro Engineering, USA).

It is further possible, that in the process of the present invention a so-called multiple compaction is carried out. In this case the particles resulting from step (iii) are recycled into the compaction step (ii). Optionally, further excipients can be added during each cycle. Preferably, 2 to 5, more preferably 3 to 4 cycles are carried out.

In a preferred embodiment the granulation conditions are chosen such that the resulting granulated pharmaceutical composition comprises a volume mean particle size (D₅₀) of 10 to 1000 μm, more preferably of 20 to 800 μm, further more preferably of 50 to 700 μm, most preferably of 100 to 650 μm. The volume mean particle size (D₅₀) is determined by the light scattering method, using a Mastersizer 2000 apparatus made by Malvern Instruments (wet measurement, 2000 rpm, ultrasonic waves for 60 sec., data interpretation via Fraunhofer method).

The bulk density of the granulated pharmaceutical composition made by the process of the first embodiment generally ranges from of 0.2 to 0.85 g/ml, preferably of 0.25 to 0.85 g/ml, more preferably of 0.3 to 0.8 g/ml.

The granulated pharmaceutical composition of the invention made by the process of the first embodiment preferably possesses Hausner ratios in the range of 1.05 to 1.6, preferably of 1.06 to 1.4, more preferably between 1.08 to 1.3. The Hausner ratio is the ratio of tapped density to bulk density.

Step (iv) comprises compressing the mixture into tablets. If the process of the present invention is carried out as direct compression, then the mixture of step (i) is compressed. Preferably, the process of the present invention is carried out as dry granulation. In this case, the mixture resulting from step (iii) is compressed.

Generally, further excipients may be added in the compression step, wherein the amounts of above-mentioned further excipients which are employed in the compression step depend on the amounts of excipients which have already been employed in the process step (i) (or alternatively, in the process steps (ii) or (iii)). For example, if the final tablet core should comprise 30% binder, it would be possible to add 20% binder before the compaction step (ii) and 10% binder before the compression step (iv) or e.g. alternatively 25% binder before the compaction step (ii) and 5% binder before the compression step (iv).

The compression step (iv) is preferably carried out with a rotary press, e.g. on a Fette 102i (Fette GmbH, Germany).

If a rotary press is applied, the main compaction force usually ranges from 1 to 50 kN, preferably from 2 to 40 kN, more preferably from 2.5 to 35 kN.

The tablets of the present invention are covered with one or more release determining layers comprising preferably components (d) and (e) or alternatively comprising component (d) or alternatively comprising components (d) and (f) or alternatively comprising components (d), (e) and (f).

Preferably, the shell of the tablet is capable of increasing the dissolution time of the pharmaceutical composition at least four-fold, more preferably at least eight-fold, according to USP release method using apparatus 2 (paddle), compared to the same pharmaceutical composition without the release modifying coating.

The shell of the tablets of the present invention is applied in process step (v). Said step comprises coating the tablet core with a coating comprising preferably compounds (d) and (e) or alternatively comprising component (d) or alternatively comprising components (d) and (f) or alternatively comprising components (d), (e) and (f).

The coating process is generally carried out in a continuously process in a pan coater or a fluid bed dryer.

The coating process is preferably carried out on a pan coater, e.g. on a Lodige LHC 25 (Lödige GmbH, Germany).

If a pan coater is applied, the spray pressure usually ranges from 0, 8-2 bar, preferably from 1 to 1.5 bar.

The product temperature varies according to the applied polymer. Usually the product temperature is adjusted by 20-40° C., preferably from 32-38° C.

The coating usually has a thickness of 0.01 to 2 mm, preferably from 0.1 to 1.5 mm, more preferably from 0.2 to 1 mm.

In a particularly preferred embodiment the core of the tablet of the present invention can be prepared by a melt granulation or melt coating process, wherein Compound I (component (a)) preferably is dispersed with at least one solubilizer, optionally a pseudo-emulsifier and optionally a pharmaceutically acceptable carrier or matrix by a melting (fusion) process, i.e. Compound I is granulated with a melted mass of excipients. After cooling, the obtained mass is preferably granulated, i.e. for example crunched, grinded and sieved and finally compressed to tablets. Alternatively, the melted mass can be charged directly in a mold to give tablets. In this embodiment preferably only polymeric solubilizers (b) are used.

Hence, a further subject of the present invention is a process for producing a tablet core as described above, comprising the steps of
(i) mixing a compound (a), (b) and optionally (c) and/or further polymeric excipients,
(ii) melting the mixture, wherein the melting conditions are chosen such that component (a) remains in crystalline form I,
(iii) cooling off (if necessary) and granulating the melted mixture.

In step (i) the compound according to formula I (=Compound I) is mixed with excipients. Preferably, the excipients comprise a solubilizer and a pseudo-emulsifier. Generally, it is noted that all comments made above regarding the solubilizer (b) and the pseudo-emulsifier (c) of the pharmaceutical composition of the present invention also apply for the processes of the present invention. However, in this embodiment preferably only polymeric solubilizers (b) are used.

Optionally, also a carrier or matrix, employing the following polymeric material, can be used: derivatives of cellulose, sugar alcohols, derivatives of organic acids, derivatives of fatty acids, waxes, semi-synthetic derivatives of glycerol.

For the melt granulation, for example, an extrusion process or high shear process may be used. The melting conditions are preferably chosen such that the active ingredient remains in crystalline form I.

The obtained complex is in step (iii) granulated (that means for example crunched, grinded and sieved) in a third step, preferably by any sieving machine, e.g. Comil® U5.

In a preferred embodiment the granulation conditions are chosen such that the resulting granulated pharmaceutical composition comprises a volume mean particle size (D₅₀) of 10 to 500 μm, more preferably of 20 to 400 μm, further more preferably of 50 to 300 μm, most preferably of 50 to 200 μm. The volume mean particle size (D₅₀) is determined by the light scattering method using a Mastersizer 2000 apparatus made by Malvern Instruments.

The bulk density of the granulated pharmaceutical composition made by the process of the fourth embodiment generally ranges from of 0.2 to 0.85 g/ml, preferably of 0.25 to 0.85 g/ml, more preferably of 0.3 to 0.75 g/ml.

The granulated pharmaceutical composition of the invention made by the process of the fourth embodiment preferably possesses Hausner ratios in the range of 1.05 to 1.6, preferably of 1.08 to 1.4, more preferably between 1.10 to 1.3. The Hausner ratio is the ratio of tapped density to bulk density.

As mentioned above, different processes are suitable for preparing the tablet comprising core and release modifying shell of the present invention.

In an alternative embodiment the pharmaceutical composition of the present invention can be prepared as a release modified composition in particulate form by a pellet layering process.

Hence, a further subject of the present invention is a process for producing a pharmaceutical composition, comprising the steps of
(i) providing a pellet core,
(ii) providing a solution or suspension comprising the components (a), (d) and preferably (e), and optionally (b), (c) and/or further excipients,
(iii) spraying the solution or suspension onto the pellet core, and
(iv) optionally blending the pellets with components (b) and (c) and/or further excipients.

In this pellet layering embodiment, the present invention provides a process for the manufacture of a pharmaceutical composition comprising Compound I, employing a pellet layering process. Herein Compound I (=component (a)) is dispersed in a solution or dispersion of one or more pharmaceutically acceptable excipients. This solution or suspension is sprayed onto an inert core, which is preferably made from water soluble or insoluble materials. In a preferred embodiment of this process component (b) is employed in any case, that means component (b) is employed in step (ii) or in step (iv) on in steps (ii) and (iv).

In step (i) a pellet core is provided. Preferably, the pellet core is a so-called neutral pellet core, that means it does not comprise an active ingredient. The pellet core can be made of suitable materials, e.g. cellulose, sucrose, starch or mannitol or combinations thereof. In a preferred embodiment the pellet core comprises or consists of one or more solubilizer(s) (b) as defined above.

Solubilizers used for the pellet core might be selected from derivatives of cellulose (hydroxyproplymethyl cellulose (HPMC), hydroxypropyl cellulose (HPC), hydroxyethyl cellulose), polyvinylpyrrolidone, copolymers of polyvinylpyrrolidone (Povidon® VA 64; BASF), polyoxyethylene-alkylethers, polyethylene glycol, sugar alcohols, like isomalt, sorbitol or mannitol, block copolymers of ethylene oxide and propylene oxide (Poloxamer).

In addition, the pellet core may comprise an osmotic agent as for example organic or inorganic compounds just as PEG or NaCl.

Suitable pellet cores are commercially available under the trade name Cellets® and preferably comprise a mixture of lactose and microcrystalline cellulose.

Furthermore, in a preferred embodiment pellet cores, commercially available as Suglets®, are used. Those preferred pellet cores comprise a mixture of corn starch and sucrose. The mixture usually comprises 1 to 20 wt. % corn starch and 80 to 99 wt. % sucrose, in particular, about 8 wt. % corn starch and 92% sucrose.

In step (ii) the compound according to formula I (=Compound I) is dissolved or suspended in a solvent. The solvent can be water, a pharmaceutically acceptable organic solvent or mixtures thereof. Preferably, the solvent is water or an alcohol. Most preferably, the solvent is water.

The solution or dispersion of Compound I can comprise further excipients. It preferably comprises a solubilizer (b) and/or a pseudo-emulsifier (c). Generally, it is noted that all comments made above regarding the solubilizer (b) and the pseudo-emulsifier (c) of the pharmaceutical composition of the present invention also apply for the processes of the present invention. In addition, the solution or dispersion may comprise anti-sticking agents and lubricants. Reference is made to the explanations given above for the first embodiment of the process of the present invention.

The solution or dispersion further comprise one or more non-erodible polymers (d). Preferably, a non-erodible polymer as illustrated above is used.

The solution or dispersion further comprises one or more pore-forming substances (e), which are also illustrated above.

The solution or dispersion further comprises one or more plasticizer(s) (f), which are also illustrated above.

In the third step (iii) the emulsion or suspension is sprayed onto the pellet core, preferably by an fluid bed dryer, e.g. Glatt GPCG 3 (Glatt GmbH, Germany).

In a preferred embodiment the spraying conditions are chosen such that the resulting particulate pharmaceutical composition comprises a volume mean particle size (D50) of 10 to 1000 μm, more preferably of 20 to 800 μm, further more preferably of 100 to 750 μm, most preferably of 250 to 650 μm. The volume mean particle size (D50) is determined by the light scattering method using a Mastersizer 2000 apparatus made by Malvern Instruments.

The bulk density of the particulate pharmaceutical composition made by the process of the second embodiment generally ranges from of 0.2 to 0.85 g/ml, preferably of 0.25 to 0.85 g/ml, more preferably of 0.4 to 0.85 g/ml.

The particulate pharmaceutical composition of the invention made by the process of the second embodiment preferably possesses Hausner ratios in the range of 1.05 to 1.6, preferably of 1.08 to 1.4, more preferably between 1.08 to 1.3. The Hausner ratio is the ratio of tapped density to bulk density.

Said processes lead to pharmaceutical compositions in granulate form. Therefore, a further subject of the present invention are granulates (=particles) obtainable by any of the processes of the present invention. These granules can be regarded as a so-called “primary pharmaceutical composition”. Depending on the nature of the polymers used in the production of the granules also primary pharmaceutical compositions having modified release properties can be obtained.

Regarding the terms “granulates” and “granulate form”, it is noted that within this application these terms refer to any particulate form of the (primary) pharmaceutical composition. Preferably, the granules have mean diameters as mentioned above. That means, that the terms “granulates” and “granulate form” may also cover particles which are in the art sometimes referred to as “pellets”.

Alternatively, the pellet layer process as described above could be modified. In this modified embodiment in a first spraying step components (a), (b) and optionally (c) are applied and subsequently in a second spraying step components (d) and (e) are applied. Hence, the present invention refers to a process for producing a pharmaceutical composition, comprising the steps of
(i) providing a pellet core,
(ii-1) providing a solution or suspension comprising the components (a), (b) and optionally (c) and/or further excipients,
(iii-1) spraying the solution or suspension resulting from step (ii-1) onto the pellet core,
(ii-2) providing a solution or suspension comprising the components (d), preferably (e), and optionally (c), (f) and/or further excipients,
(iii-2) spraying the solution or suspension resulting from step (ii-2) onto the pellets resulting from step (iii-1), and
(iv) optionally blending the pellets with components (b) and (c) and/or further excipients.

The granulates of the present invention (i.e. the primary pharmaceutical composition) may be used to prepare suitable solid oral dosage forms with modified released properties. That means, the primary pharmaceutical composition can be further processed to give a “final pharmaceutical composition”, i.e. to give a final oral dosage form.

Hence, the present invention encompasses a process for producing oral dosage forms comprising a pharmaceutical composition as received by the above-described pellet layering process, comprising the steps of
(i) optionally mixing the granulates as received by the above-described pellet layering process with further excipients,
(ii) further processing the resulting mixture into a final oral dosage form.

Preferably, step (ii) comprises
(ii-α) filling the resulting mixture into capsules
(ii-β) filling the resulting mixture into sachets or
(ii-γ) compressing the resulting mixture into tablets.

That means, the granulates can be compressed to a tablet or filled into capsules or sachets, optionally after blending with other excipients. A particularly preferred dosage form is in the form of tablets.

The modified release formulations of the present invention (i.e. the pharmaceutical composition, the tablet comprising core and shell and the dosage forms obtained by the pellet layering process) comprise the following types of drug release:

The modified release formulation might be a sustained release type which provides an initial starting dosage high enough to set on the pharmaceutical effect and which sustains this pharmaceutically optimal dosage for a certain period of time longer than achievable by applying a normal single dose medication.

The modified release formulation might be a prolonged-release type, which releases an initial starting dose, being sufficient but not unacceptable high. The starting doses provides the required pharmaceutical effect and the formulation furthermore releases continuously enough drug resulting in a measurable increase of time where the action of the drug takes place.

The modified release formulation might be a repeat-release type or staggered-release type, which provides a first initial starting dose and which subsequently releases one or more additional single dosages.

The modified release form might be a delayed release type, which releases the dose only after a certain period of time after administration of the dosage form.

In any case can the final dosage form also combine two or more of the above mentioned modified release types.

Furthermore, modified release formulations of the present invention (i.e. the pharmaceutical composition, the tablet comprising core and shell and the dosage forms obtained by the pellet layering process) preferably show an in vivo drug release profile of zeroth or first order.

The dosage forms of the present invention (preferably the tablets) may contain dosage amounts of 1-120 mg, preferably 5-60 mg, more preferable 10-50 mg, e.g. 10 mg, 20 mg, 25 mg or 50 mg of the active pharmaceutical ingredient. Thus the administered amount can be readily varied according to individual tolerance and safety warranting a flexible dosing.

The tablets of the present invention preferably have a friability of less than 1%. Furthermore, the tablets of the present invention preferably have a hardness of 60 to 200 N, more preferably from 70-150 N.

Finally, subjects of the present inventions are tablets obtainable by any of the processes as described above.

In another aspect, the present invention provides the use of the pharmaceutical composition of the present invention for the prophylaxis and/or treatment of thrombo-embolic diseases, such as infarct, angina pectoris (including instable angina) re-occlusions and restenoses after an angioplasty or an aorta-coronary bypass, stroke, transitory ischaemic events, peripheral arterial occlusion, lung embolism or deep vein thrombosis.

Where it is referred to the total weight of the pharmaceutical composition and the pharmaceutical composition in a single dosage form, the total weight is the weight of the single dosage form excluding, if applicable, the weight of any coating or capsule shell.

The invention is now illustrated in the following examples, which are not to be constructed as being limiting.

### EXAMPLES

### Example 1

| | | |
|---|---|---|
| Rivaroxaban, micronized: | 40 | mg |
| Gum arabicum: | 3 | mg |
| Pluronic ®: | 4 | mg |
| Ethylcellulose: | 15 | mg |
| PEG 4000: | 4 | mg |
| Cellets ®: | 40 | mg |
| Microcellac ®: | 200 | mg |
| Povidon ®: | 10 | mg |
| Lubritab ®: | 5 | mg |
| Aerosil ®: | 2 | mg |
| Opadry ®: | 2.5 | mg |

### Procedure:

Compound I was suspended together with ethyl cellulose in an aqueous solution of Pluronic®, gum arabicum and PEG. The placebo pellets were pre-heated to 38° C. in a fluid bed dryer. Subsequently the pellets were coated with the suspension using the following parameter:

| | | |
|---|---|---|
| Inlet temperature: | 40-80° | C. |
| Product temperature: | 35-40° | C. |
| Spray nozzle: | 1-2 | mm |
| Spray pressure: | 1-2 | bar |

After sintering at elevated temperature the pellets were blended with Microcellac® and Aerosil® and Povidon® for 25 min in a tumble blender. Afterwards Lubritab® was added and the blend was mixed for additional 3 minutes.

The final blend was compressed on a Fette 102 I rotary press characterized by following parameter: hardness 80-110 N; Friability less than 1%.

The tablets were coated in order to achieve a better compliance with a aqueous solution of Opadry (Colorcon):

Product temperature: 37-40° C.
Supply air temperature: 40-80° C.
Nozzle diameter: 1,2 mm
Spray pressure: 1-3 bar

Afterward the tablets were sintered by 60° C. for 0,5 hour.

### Example 2

| | | |
|---|---|---|
| Rivaroxaban, co-precipitate: | 120 | mg |
| Agar: | 4 | mg |
| talcum: | 12 | mg |
| Ludipress ®: | 100 | mg |
| magnesium stearate: | 2 | mg |
| Aerosil ®: | 1 | mg |
| cellulose acetate: | 14 | mg |
| PEG 4000: | 5 | mg |
| talcum: | 1 | mg |
| pigment: | 1 | mg |
| titan dioxide: | 0.2 | mg |

### Procedure:

The Rivaroxaban co-precipitate was produced by precipitation of Compound I with hydroxypropyl cellulose in a ratio of 1:9 and SDS in a mixture of acetic acid and ethanol. Water as anti-solvent was added with stirring. The precipitate was dried at elevated temperatures. The co-precipitate was pre-blended with agar and Talcum. The obtained Co-precipitate granules were blended with, Ludipress® and Aerosil® for 30 min on a tumble blender, (e.g. Turbula TC 10 B). Subsequently magnesium stearate was added. The final blend was mixed for 3 min and compressed on a rotary press. The tablets has a friability of less than 1% and a hardness of 70-120 N. The tablets were coated with an suspension of cellulose acetate, PEG, titan dioxide and talcum in a pen coater, for example Lödige:
Product temperature: 30-40° C.
Supply air temperature: 40-80° C.
Nozzle diameter: 1.2 mm
Spray pressure: 1-3 bar

Afterward the tablets were sintered by 60° C. for 2 hours.

1. Pharmaceutical composition comprising
(a) a compound according to formula I as active ingredient 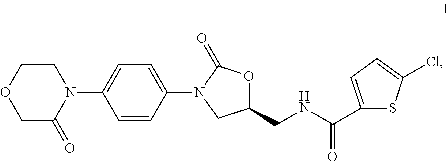
its solvates, hydrates and/or pharmaceutically acceptable salts, preferably in crystalline form,
(b) a solubilizer, preferably water-soluble compound as solubilizer having a water solubility of more than 10 mg/l at a temperature of 25° C.,
(c) optionally a pseudo-emulsifier, preferably a natural gum,
(d) a non-erodible polymer, preferably a non-erodible polymer having a water solubility of 10 mg/l or less at a temperature of 25° C.
(e) a pore-forming substance, preferably having a water solubility of more than 100 mg/l at a temperature of 25° C.

2. Tablet comprising a pharmaceutical composition according to claim 1, characterized in that the tablet comprises a core and a shell, wherein the core comprises components (a), (b) and optionally (c) and wherein the shell comprises components (d) and (e).

3. Process for producing a tablet according to claim 2, comprising the steps of
(i) mixing a compound (a), (b) and optionally (c) and/or further excipients,
(iv) compressing the mixture into tablets, and
(v) coating the tablets with a coating comprising compounds (d) and (e).

4. Process according to claim 3, wherein components (a) and (b) are employed in the form of an intermediate, which is obtained by blending of compounds (a) and (b).

5. Process according to claim 3, wherein components (a) and (b) are employed in the form of a co-precipitate, obtained by a process comprising the steps
(α) dissolving components (a) and (b) in a solvent,
(β) precipitating a complex comprising components (a) and (b) by adding an anti-solvent.

6. Process according to claim 3, further comprising the steps
(ii) dry-compaction of the mixture resulting from step (i) to give a comprimate, and
(iii) granulating the comprimate and optionally adding further excipients.

7. Process according to claim 3, wherein component (a) is employed in crystalline form, further comprising the steps of
(ii) melting the mixture, wherein the melting conditions are chosen such that component (a) remains in crystalline form I, and
(iii) cooling off and granulating the melted mixture.

8. Process for producing a pharmaceutical composition according to claim 1 in particulate form, comprising the steps of
(i) providing a pellet core,
(ii) providing a solution or suspension comprising the components (a), (d) and (e) and optionally further excipients,
(iii) spraying the solution or suspension onto the pellet core, and
(iv) optionally blending the pellets with components (b) and (c) and/or further excipients.

9. Process for producing oral dosage forms comprising a pharmaceutical composition according to claim 8, comprising the steps of
(i) optionally mixing the granulates according to claim 8 with further excipients,
(ii) further processing the resulting mixture into a final oral dosage form.

10. Process according to claim 9, wherein step (ii) comprises
(ii-α) filling the resulting mixture into capsules
(ii-β) filling the resulting mixture into sachets or
(ii-γ) compressing the resulting mixture into tablets.

11. Oral dosage forms, obtainable by a process as described in claim 3.

12. Pharmaceutical composition according to claim 1, showing sustained release, prolonged release, repeat-release and/or delayed release.

13. Pharmaceutical composition according to claim 1, showing an in vivo drug release profile of zeroth or first order.

14. Pharmaceutical composition according to claim 1, comprising a plasticizer (f).

15. Process according to claim 3, wherein component (d) is used together with a plasticizer (f), wherein component (f) is used in an amount of 1 to 30 wt. %, based on the combined weight of components (d) and (f).

16. Tablet according to claim 2, showing sustained release, prolonged release, repeat-release and/or delayed release.

17. Oral dosage forms according to claim 11, showing sustained release, prolonged release, repeat-release and/or delayed release.

18. Tablet according to claim 2 showing an in vivo drug release profile of zeroth or first order.

19. Oral dosage forms according to claim 11 showing an in vivo drug release profile of zeroth or first order.

20. Tablet according to claim 2 comprising a plasticizer (f).
